(19) United States
(12) Patent Application Publication
(10) Pub. No.: US 2007/0055146 A1
(43) Pub. Date: Mar. 8, 2007

(54) Microwave based monitoring system and method

(76) Inventors: Sebastian John Corlette, New South Wales (AU); Jon Gordon Ables, New South Wales (AU); David William Bishop, New South Wales (AU)

Correspondence Address: NIXON & VANDERHYE, PC, 901 NORTH GLEBE ROAD, 11TH FLOOR, ARLINGTON, VA, 22203, US

(21) Appl. No.: 10/552,473

(22) PCT Filed: Apr. 8, 2004
(86) PCT No.: PCT/AU04/00465

§ 371 Date: Oct. 7, 2005

(30) Foreign Application Priority Data
Apr. 8, 2003 (AU) 2003901660

(51) Int. Cl. A61B 5/05

(52) U.S. Cl. 600/430

(57) ABSTRACT

A device for monitoring fluctuations in an opaque body, the device including: (a) at least one low power microwave emitter for locating adjacent the opaque body; (b) a microwave detector for detecting fluctuations in the scattering characteristics from the opaque body; (c) a signal processing means for analysing fluctuations from the body so as to thereby derive characteristics about the body.

### FIELD OF THE INVENTION

The present invention relates to monitoring systems for monitoring the human body or the like. In particular, the present invention discloses a system for microwave monitoring of physiological parameters within the human body.

### BACKGROUND OF THE INVENTION

Many different methods have been developed for monitoring the human body or for monitoring activities within other structures. For example, pulsed or continuous wave Doppler ultrasound is often utilised to monitor the human body. Alternatively, electrical activity within the body can be monitored utilising an electrocardiograph.

It would be desirable to provide for an alternative form of transcutaneous monitoring of functions within bodies such as within the human body.

### SUMMARY OF THE INVENTION

It is an object of the present invention to utilise microwave scattering properties so as to provide for the monitoring of internal portions of bodies.

In accordance with a first aspect of the present invention, there is provided a device for monitoring fluctuations in an opaque body, the device including: (a) at least one low power microwave emitter for locating adjacent the opaque body; (b) a microwave detector for detecting fluctuations in the scattering characteristics from the opaque body; (c) a signal processing means for analysing the fluctuations from the body so as to thereby derive characteristics about the body.

In one embodiment, the emitter and detector are preferably formed as one unit. The opaque body can comprise a human body and the signal processing means extracts a heart rate from the fluctuations or the respiration rate from the fluctuations. The device can be portable and located near the chest of the human.

In accordance with a further aspect of the present invention, there is provided a method of monitoring fluctuations in the density of an opaque body, the method comprising the steps of: (a) locating a low power microwave emitter adjacent the opaque body; (b) monitoring the scattering properties of the opaque body so as to produce a monitor signal; (c) utilising fluctuations in the monitor signal over time to infer fluctuations in the opaque body.

The body can comprise a human body and fluctuations can include alterations in the blood flow rate or in the respiration rate in the human body. The low power microwave emitter can be located adjacent to the chest of the human body and can have one or two emission/reception points depending on requirements.

In accordance with a further aspect of the present invention, there is provided a remote monitoring system for monitoring a series of patients at remote locations, the monitoring systems including: (a) a series of portable monitoring units for monitoring fluctuations in a human, the monitoring units including at least one low power microwave emitter for locating adjacent the human body, a microwave detector for detecting in the scattering characteristics from the human body; a signal processing means for analysing the fluctuations from the body so as to thereby derive characteristics about the body, and a wireless communications interface for communication characteristics about the body with a spatially separated base station; (b) a series of base stations, each further interconnected with an information distribution network, the base stations receiving the characteristics from the portable monitoring units and forwarding them to a centralised computing and storage resource; (c) a centralised computing and storage resource for storing and monitoring the characteristics.

The system further preferably can include analysis means for analysing the characteristics for predetermined behaviours and raising a notification alarm upon the occurrence of the predetermined behaviours.

### BRIEF DESCRIPTION OF THE DRAWINGS

Preferred and other embodiments of the present invention will now be described with reference to the accompanying drawings in which:

FIG. 1 illustrates a first microwave sampling device;

FIG. 2 illustrates a second microwave sampling device;

FIG. 3 illustrates schematically the arrangement of the preferred embodiment;

FIG. 4 illustrates schematically the internal form of monitoring unit of the preferred embodiment;

FIG. 5 is a graph of the resulting trace data of measurements taken;

FIG. 6 is a power spectrum of the data of FIG. 5;

FIG. 7 illustrates schematically an alternative embodiment;

FIG. 8 illustrates an example of monitoring interface;

FIG. 9 illustrates a heart rate monitor; and

FIG. 10 illustrates a monitor status interface.

### DESCRIPTION OF PREFERRED AND OTHER EMBODIMENTS

In the preferred embodiment, a system is proposed for measuring bodily functions such as heart and respiratory rates. The measurements are conducted by categorising the scattering parameters of the body at microwave frequencies. The preferred embodiment utilised the microwave scattering parameters of a device to derive the physiological parameters.

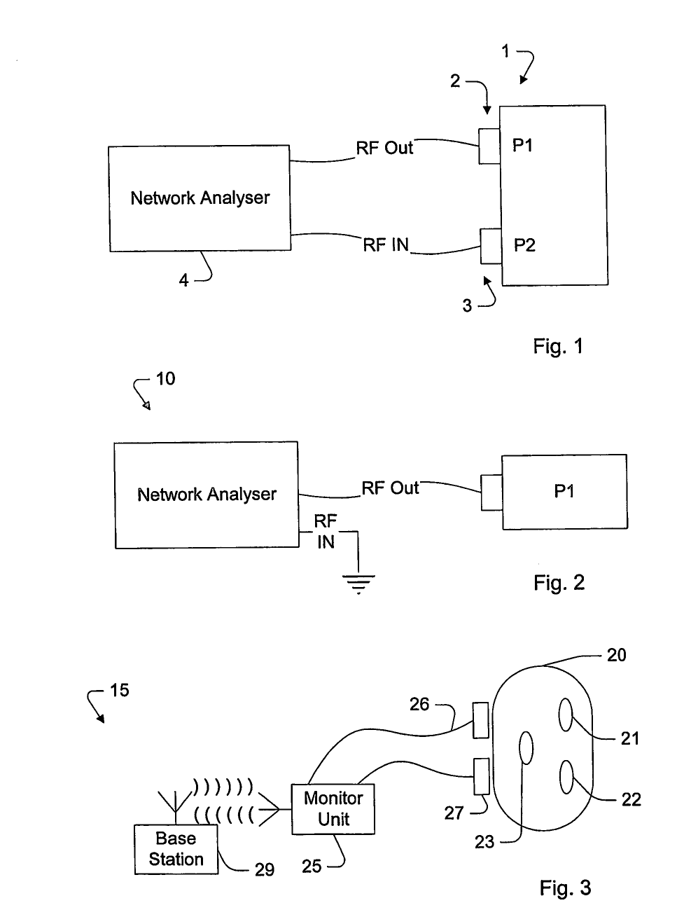

Turning initially to FIG. 1, there is illustrated schematically a method for determining the microwave scattering parameters of an arbitrary device 1 which includes two ports 2, 3. The device 1 can comprise any component that has two ports. Often the device under test can be a complex device like an amplifier or a filter. A network analyser 4 is utilised to emit microwave radiation frequencies to the port P1 and the RF input is measured at port P2. For the two port device 1 there are normally four parameters denoted s₁₁, s₁₂, s₂₁, s₂₂ which identify the scattering parameters. These are in general complex numbers, that is, having both magnitude and phase. The subscripts refer to the ports (port 1 and port 2). S_{ab} is the voltage phasor at port a due to excitation at port b by a voltage with unit phasor (magnitude=1, phase=0). Port 1 is usually (but not necessarily) the designated input of the device and port 2 is the output. Thus s₂₁ for an amplifier is its overall complex gain amplification-factor and phase-shift.

The same concept can be used for a simple, one-port device as illustrated 10 in FIG. 2. In this case there is only one scattering parameter, s₁₁. Here s₁₁ is the complex amplitude of the microwave energy flowing back out of the input port P1 due to energy flowing into the device.

In the preferred embodiment, the arrangements of FIG. 1 and FIG. 2 are utilised to measure physical parameters inside the human body. The arrangement is illustrated schematically in FIG. 3 wherein the schematic sectional view of human body 20 includes lungs 21, 22 and heart 23. A low power microwave frequency monitoring unit 25 is provided having one or two couplers 26, 27 which couple to the human body. The couplers are placed close to the body without actually touching it.

The coupling is effected through electric (E) or magnetic (H) fields or a combination of both. The dominant mode of the EH field will be the so-called induction (near) field which, at very close range, is much stronger than the radiation (free propagation) field. Since the sensor relies on the induction field, it is inappropriate to designate these couplers as antennas, just as the input coupling capacitor (which is a pure E-field device) of an audio amplifier is not an antenna Both two-port and one-port implementations of the sensor can be realised. The one-port version, by requiring only one coupler, is the more compact realization.

Heartbeat and respiration cause the microwave scattering parameters of the body (primarily the thorax) to be time dependent. Measurements of the appropriate scattering parameter, as a function of time, shows variations in both magnitude and phase from which useful measures of heart and lung function can be extracted. Even the most simple of these, the beat-to-beat and breath-to-breath intervals, can be very valuable for determining the well being of a subject.

The monitor unit, through the replacement of the laboratory instrument network analyser with a microcircuit equivalent, is capable of being small enough and low power enough to be used as a wearable, battery-powered, continuous monitor of the cardio-pulmonary status of a subject living away from medical high-care facilities. The monitor unit 25 is interconnected via wireless communication to a base station 29.

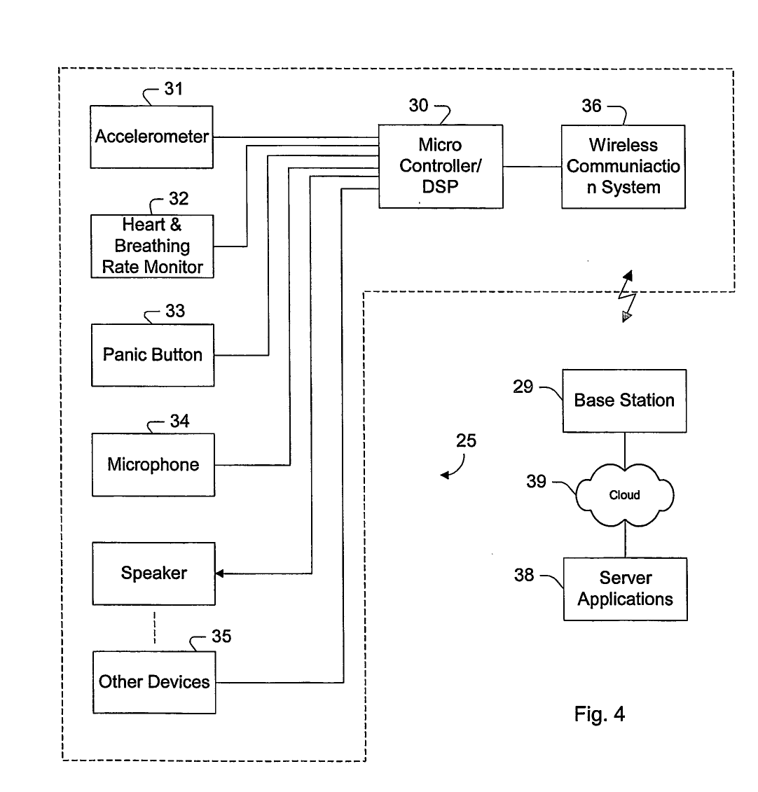

Turning now to FIG. 4, there is illustrated in more detail the schematic arrangement of one form of monitor unit 25. The monitor unit 25 can be based around a core microprocessor/micro controller 30 which has interconnected to it a series of inputs in the forms of an accelerometer 31, a heart and breathing rate monitor 32, a panic button 33, a microphone 34 and other devices e.g. 35 that may be desirably required. The microcontroller 30 can include on board digital signal processing capabilities and is interconnected to a wireless system 36 for communicating with a base station 29. The base station 29 can in turn be interconnected with a server device 38 over an Internet type arrangement 39.

A microwave monitoring device was constructed in accordance with the aforementioned guidelines so as to monitor heart rate and respiratory rate and other activities such as movement and orientation. The microwave radio transmission was at 915 megahertz which enabled detection of bodily movement via near field variations on the couplers 26, 27 of FIG. 3.

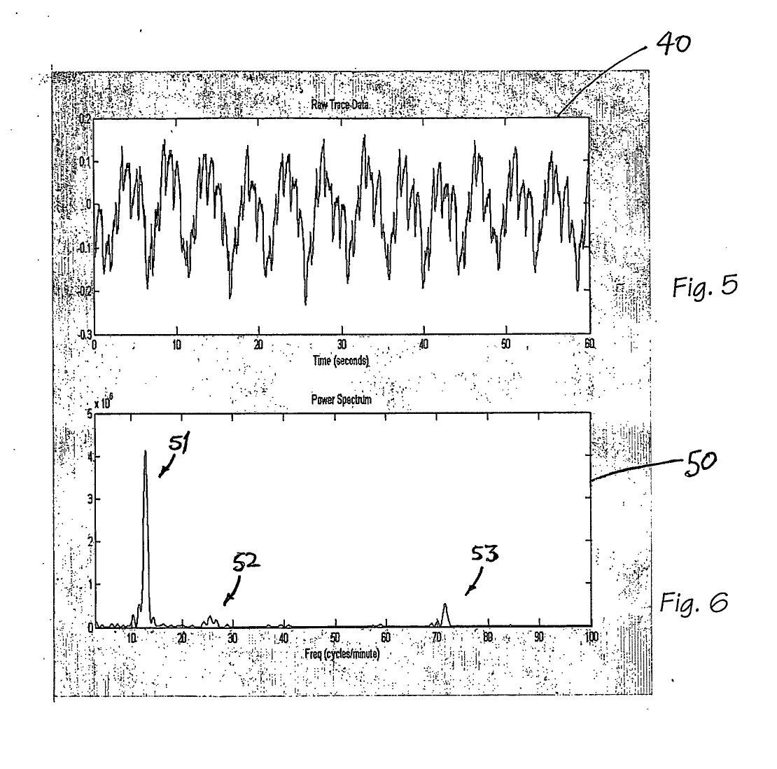

FIG. 5 illustrates the resultant raw trace data 40 obtained. It can be seen to have a substantially periodic nature. FIG. 6 illustrates the corresponding power spectrum for the arrangement of FIG. 5. Analysis of the spectrum reveals a series of peaks 51-53. The peak 51 was found to correspond to a fundamental respiration peak. The peak 52 was found to correspond to the second harmonic of the respiration peak. The peak 53 was found to correspond to the wearer's heart rate.

The system 15 of FIG. 3 is able to collect selected vital signs from a participating user. If any of the collected parameters indicate a potentially critical situation, a software alarm can be raised to allow the appropriate clinicians, family members etc to be notified. Data can be collected from a number of participants including the healthy. A database of clinical results can be stored to enable future assessment of the client's health in addition to investigation of statistical parameters across a population. The user-worn monitoring unit 25 can collect the vital sign parameters and perform some analysis and summarization. The data from the non-contact sensors, which can be located in the client's pocket, can be transmitted to a server via a mobile or conventional phone.

The information that can be transmitted to a host system can include: Activity data, Heart rate, Respiration rate, Temperature, Battery voltage, A panic button alarm, Proximity to body alarm, Low battery alarm, Fall alarm, and Microphone and Loudspeaker signals to allow interaction with client

The signals are collected from the sensors and are processed by the microcontroller 30 before being sent to a central database. The processing can vary in its complexity and the resultant data can be transmitted under certain defined criteria. The device in itself can have various modes of operation. This table describes example modes of operation that the module can have.

| Mode | Description |
|---|---|
| 1 | Device is turned off. (Inferred from the fact that it is not in mode |
| | of operation 2 or 3). |
| 2 | Device is turned on and not in proximity to a body. |
| 3 | Device is turned on and the device is in close proximity to the |
| | body. In this mode the system generates valid data. |

Data can be collected from the accelerometers and can be simplified into a number, which best represents the activity of the wearer. This number can be transmitted to the central computer system immediately if a fall has been detected. Otherwise should the subject state change (reported on exception) it can be stored in a local buffer in the microcontroller. The accelerometer states can be as follows:

| State | State Description | Value |
|---|---|---|
| 1 | No movement of the subject. | 10 |
| 2 | The subject is walking. | 100 |
| 3 | The subject is engaged in vigorous activity. | 1000 |
| 4 | The subject has fallen down. | 1 |

A time interval can precede this number. This interval is added to the initial time transmitted at the start of each buffer transmission to form an absolute time. Should a suspected fall occur, an alarm bit is set and the device operates in an alert manner and sends data from the client to the central monitoring system for the next 5 minutes. This allows the operator to analyse the activity of the wearer to determine if they have recovered from the suspected fall. In a similar manner to the accelerometer data, the respiration and heartbeat R-R measurements are collected and stored in a local buffer in the microcontroller.

The battery voltage can also be measured and regularly transmitted to the host server. The time period of transmission can be say every 30 minutes.

There can be four types of priority alarms that can be generated by the Monitor Unit 25. These can include:

1. Panic Button—Whenever the subject presses the panic button 33, the data in the microcontroller's data buffer is transmitted to the host server, together with the panic button status bit.
2. Proximity to body—When the device is close to the body the proximity to the body status bit is set
3. Low battery—The battery voltage of the system is monitored, when below a minimum range, a high priority alarm is generated, to indicate that the battery in the Monitor Unit 25 needs to be either charged or changed. An LED on the Monitor Unit 25 can also be lit.
4. Fall detected—If the accelerometer detects a fall then the fall status bit is set. This allows for fast detection of the device status.

Should the operator of the host server wish to get in touch with the wearer of the device, the operator can enable the voice over IP system which can allow full duplex communication with the device wearer or the operator may send a signal to the device to broadcast aloud a prepared message which may elicit a response from the client such as getting them to press a button. Speech coding, decoding can be relatively low quality, the main criteria being that the speech is recogniizable. Using ITG G.722 speech compression with an output bit rate of 8 kbit/s steps may be suitable.

The system can be optimized to minimize power consumption. To do this the various subsystems can be shut down or placed in a sleep mode when they are not being used. Data can be collected from the accelerometers at a set interval. Preferably a three axis accelerometer can be used and signals sampled. Data can be sampled from the heartbeat/respiration sensor and processed to give the following measurements:
1. Respiration period,
2. R-R heart rate and
3. Body proximity indication.

If any of these values have been changed they can be stored in a buffer with a time interval defined. The initial time can be a value set by an onboard integrated circuit or local high accuracy clock. The Monitor Unit 25 local time can be set via a message sent by the host server. Any spare RAM located on the DSP processor can be used for buffering of the data. This can be flushed after successful transmission to the host server.

When the host server receives a packet of data from the device it can send an acknowledgement message. This can allow data to be cleared from the onboard device RAM. If the buffer becomes full to its capacity because of loss of communication with the host server, then the most recent data can be kept for transmission when communication to the host server is resumed. The amount of data packets to be stored depends on the importance of the data (certain data is prioritized higher than others when communications have failed) and the amount of time communications have failed.

Data can be transmitted to the host server using TCP/IP over a Bluetooth link. The two communication methods can be:
1. GPRS mobile phone network or
2. PSTN

The PPP layer can be coded in the microcontroller/DSP chip 30.

PSTN Modem Communications

The data flow from the sensor in the Monitor Unit 25 to the server is as follows.

```
  1. Data captured by sensor
  2. Sensor data processed in microcontroller/DSP
  3. Data sent out serially via DSP's UART
  4. Data serially into Bluetooth processor via UART
  5. Bluetooth processing in Processor in RFCOM mode
  6. Data transmitted via RF to Bluetooth receiver
  7. Data received by Bluetooth receiver
  8. Data sent out serially via UART to modem
  9. Data received at SQL server
10. Data stored in SQL server
```

GPRS Communications

The data flow from the sensor in the Monitor Unit 25 to the server is as follows.

1. Data captured by sensor
2. Sensor data processed in microcontroller/DSP
3. Data sent out serially via DSP's UART
4. Data serially into Bluetooth processor via UART
5. Bluetooth processing in processor in RFCOM mode
6. Data transmitted via RF to Bluetooth receiver
7. Data received by Bluetooth receiver in GPRS phone
8. Data received at SQL server
9. Data stored in SQL server

Data transmission from the DSP on the Monitor Unit 25 to and from the host server can be undertaken using the same data packet structure. The data packet can be of a dynamic length, whose length is only limited by the underlying network protocol used, which in this case is TCP/IP.

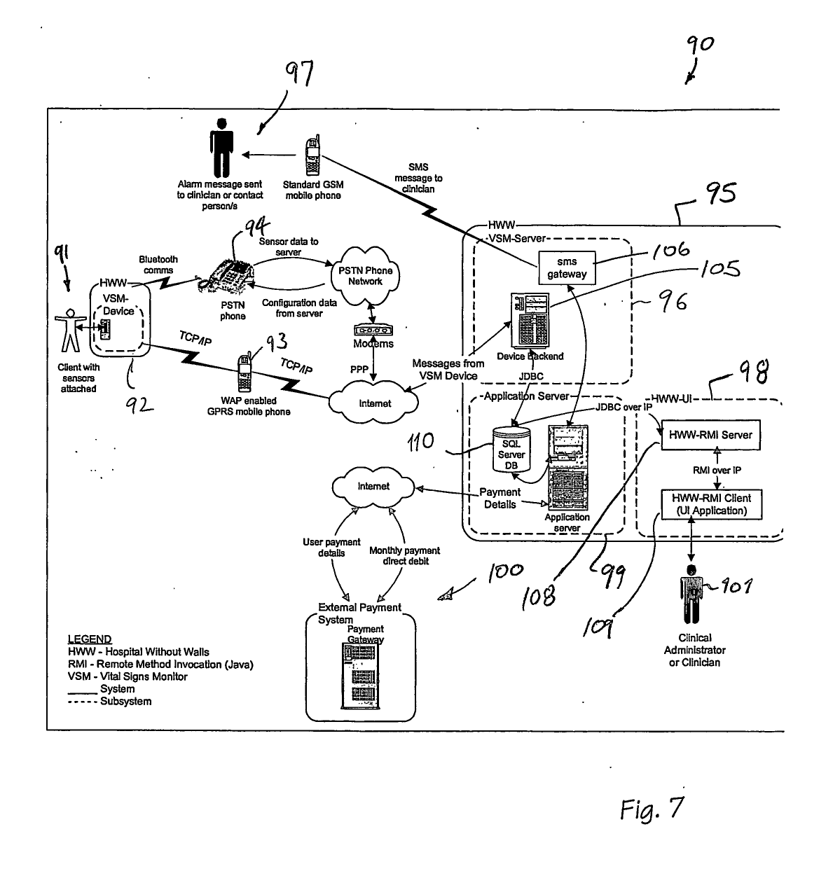

Turning now to FIG. 7, there is illustrated schematically an alternative arrangement 90 for incorporating a sensor interface to the human body. A patient 91 is fitted with the monitoring device 92 which interconnects via either a WAP enabled GPRS mobile phone 93 or a PSTN phone 94 to connect via the internet to a server system 95. The server system includes a number of servers which include a first server 96 for connecting with the monitoring devices and sending SMS messages to relevant personnel 97. A further server 98 is provided for user interface interactions with the overall servers 95 and an application server 99 stores relevant data and programs for monitoring patients in addition to interacting with other computers such as computers providing external payment services 100.

This VSM-server receives the monitor data and spools the data into the database 110. Configuration of the system provides a linkage between the address the data is emanating from (IP address) and the client's name. The five data values are stored for each client together with a time stamp. Further derived values can be added, as the system is refined.

Configuration of the system is done through an operator interface. Linkages between incoming sensor data, outgoing SMS, email data transfers and client can to be set up. This can be done from a system configuration menu.

Operators 101 may enter and view data. Data insertion can include the entry of client demographic details. This data can be linked to the incoming sensor data stream. Alarms can be set for individual client parameters. For example, “Sigh pulse rate” or “Low respiration rate”. Data collected from the real time sensors can be retrieved for viewing. This data may be in the form of a trend, alarm list or client details.

Account management allows the user to view and update account details. Each user or user's proxy will periodically be billed for use of the system via payment gateway 100. The billing functionality may be implemented by:
1. Sending out a bill.
2. Initiating a direct debit from a user's bank account.
3. Initiate a Credit card transaction.

User administration shall also be achieved. The various administration rights for the users are as follows:
Client: Data from their sensors are stored in the system.
Clinician: May add new clients, set up client demographics and retrieve client data.
Clinical Administrator: Has the ability to configure the system and can access any of the system to do anything.

The server 98 accesses data from the database server 99 and presents it to users through a standard web page. All users can access the system through this interface 98.

This application server is responsible for servicing data to and from the desktop application. When the system user sends data for storage or retrieves data, the application server processes the user request. This server provides the pipe connecting the Database with the client and performs the required processing of the data.

The GPRS or PSTN phone system sends data to the system. The server 96 takes this data and preprocesses it before storage in the database. Preprocessing can include data compression if raw data is coming from say an ECG sensor.

The database server stores all data pertaining to users of the system as well as the systems administration and configuration data. The database server can be a computer running Microsoft SQL server. This allows data structure porting to a smaller system that may be located in a home or nursing home using MSDE 2000.

The system architecture diagram of FIG. 7 provides an overview of an alternatively structured system and illustrates the various components and their interactions with each other, any external interfaces and their interaction with the system. These modules consist of both software and hardware components.

The data shall emanate from a sensor being worn in the upper left hand pocket of patient 91. The sensor includes signal conditioning electronics. The micro controller formats data and sends it to a transmitter also located in the device. This sends the data using the Bluetooth standard to a phone, nearby. The aerial for the data transmitter can be either on the sensor, sewn into the pocket or sewn into a lanyard located around the user's neck.

The number of input devices can be dependant on the data rate to be captured.

The VSM server 96 subsystem is made up of two separate components the Device Backend 105 and the SMS Gateway 106. The SMS Gateway component is implemented using Java and communicates directly to the SQL Server DB located in the Application Server subsystem 99.

Activation of the SMS Gateway component is via pre-defined triggers issued by SQL Server. These triggers parse the data sent to it by the trigger into a corresponding form of recognizable plain English text for the person communicated to.

The Device Backend component 105 is a Java application that communicates either to the client's GPRS phone or to their home phone via a PSTN network.

The HWW-UI subsystem 98 is made up of two separate components the HWW-RMI Server 108 and the HWW-RMI Client 109 application.

The subsystem 98 can be implemented using n-tier Java technology for the following benefits:—
Allows callbacks from server to client.
Preserve security as provided by the Java runtime environment.
Provides seamless remote method invocations betweens objects residing on different machines.
Distributed applications can be run easily.

A powerful side benefit is that there lies a clear distinction between remote and local objects.

The HWW-RMI contains the business logic of the system. It connects to Application Server subsystem, specifically the SQL Server DB via a JDBC connection. Multiple instances of the HWW-RMI Client applications 109 then connect to it. It receives method calls from the HWW-RMI Client application and these method calls then query the DB, a resultant returned resultSet object is then parsed into a different form, and the relevant objects or primitive data types are then returned to the top tier. It is to run continuously on a computer that is suitably robust, i.e. it has a UPS and sufficient memory resources, and bandwidth to support the component when running. This computer also has an SQL Server JDBC driver loaded on it.

This HWW-RMI Client component contains a user interface (UI) that encapsulates the functionality associated with the System Configuration and Operation areas.

This UI allows:
The clinical administrator to manage the system and other users/operators access and to view all relevant patient information.
The monitoring of trends and alarms.
Billing management.

The client application allows registered users/operators of the system to manipulate and configure it.

Access to patient's details requires a user to be registered in the system. Due to the different types of users the GUI will have different levels of functionality enabled depending on the level of access each user's needs. The 2 levels of access are:
Clinical Administrator—Manages the database and also adds, deletes and edits all the other users groups. They also monitor generated alarms and trends.
Clinician—Some type of medical professional. They can monitor the medical data coming from their associated patients.

A Patient/Client shall have no access to the web site.

As there are 2 levels of access, 2 separate applications have been created.
Hospital Without Walls (Administrator)—Only a Clinical Administrator can access this application.
Hospital Without Walls—Only a clinician or clinical administrator can access this application.

Each client can have an alarm associated with each vital sign variable, for example heart beat, respiration rate etc. These will have the classic high and low alarms. When an alarm is generated and sent by the monitor device the following operation occurs:
The alarm triggers an event that updates the DB.
If the screen is already running then an update of the display will be forced. All triggered alarms will be written to a file.

The alarm screen provides access to the DB which stores the alarms generated by the VSM device. Several options are available from the screen. These are:
Displaying alarms
Enabling/disabling alarms
Acknowledging alarms and
Configuring alarm beeping.
   Displaying alarms

There are three viewing modes for the alarm screen. They are:
1. Present alarms
2. Disabled Alarms
3. All Configured alarms

In conjunction with these viewing modes there are three types of alarms. These are:
1. Active acknowledged
2. Inactive aclnowledged
3. Inactive unacknowledged

This screen displays the following information:
Time and date that the alarm became active, alarm tag name or code, alarm name, alarm description. Alarm status and indication of whether the alarm is enabled is also provided.

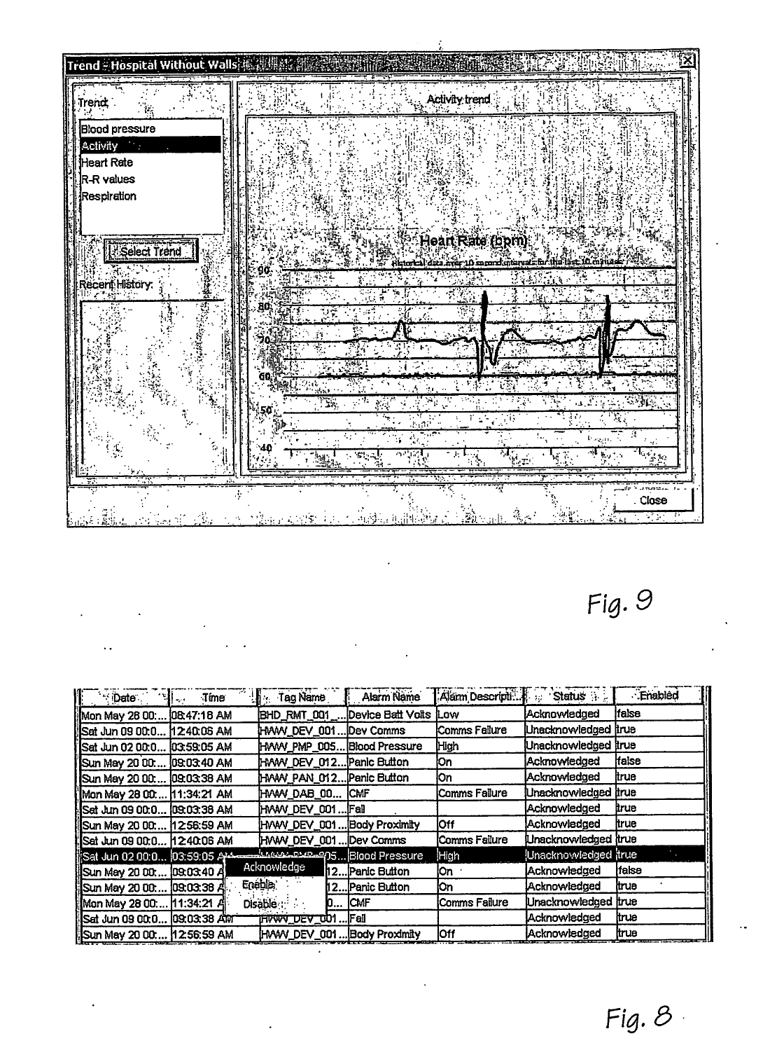

FIG. 8 illustrates an example alarm interface screen with options enabled via a popup menu.

All vital sign variables will be able to be trended. A trend is called up on the basis of the client's name and date required. FIG. 9 illustrates an example variable data output.

A multi-trend screen can be implemented with multiple dialogs appearing on screen or a single dialog with small snapshots of the trends appearing, in which the user can click on each to enlarge it and gain a better view.

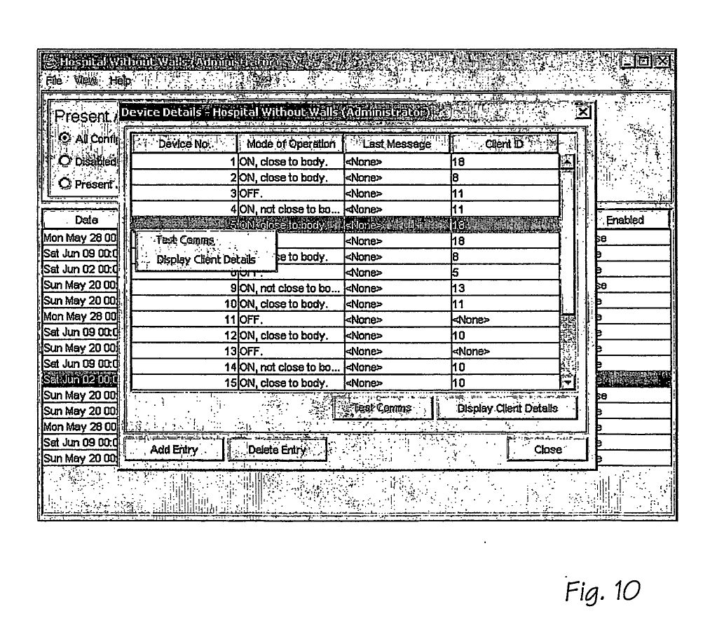

Preferably, the user interface allows for monitoring of the monitor devices that are connected to the system. An example interface is illustrated in FIG. 10 wherein the mode of operation and last message sent are displayed. The information in the table dynamically refreshes itself. Several options are available from the screen. These are:
1. Adding a new monitor unit to the system.
2. Deleting an existing device.
3. Testing communications to an individual device.
4. Displaying the details of the client who is using that particular device (if a client exists).

The screen lists all the clients in the DB. A search function is provided so that either the clinician or clinical administrator can search for a client using criteria such as, client ID, given name or surname.

One method of operation can include programming so as to notify the central server when the device is being worn. In this manner, the user can be encouraged to wear the device at appropriate times.

The foregoing describes preferred embodiments of the present invention. Modifications, obvious to those skilled in the art can be made there to without departing from the scope of the invention.

1-19. (canceled)

20. A device for monitoring fluctuations in an opaque body, the device comprising:
(a) at least one low power microwave emitter for locating adjacent the opaque body;
(b) a microwave detector for detecting fluctuations in the scattering characteristics from said opaque body;
(c) a signal processing means for analysing said fluctuations from the body so as to thereby derive characteristics about said body.

21. A device as claimed in claim 20 wherein said emitter and detector are formed as one unit.

22. A device as claimed in claim 20 wherein said opaque body comprises a human body and said signal processing means extracts a heart rate from said fluctuations.

23. A device as claimed in claim 20 wherein said opaque body comprises a human body and said signal processing means extracts a respiration rate from said fluctuations.

24. A device as claimed in claim 20 wherein said device is portable and located near the chest of the human.

25. A method of monitoring fluctuations in the density of an opaque body, the method comprising the steps of:
(a) locating a low power microwave emitter adjacent said opaque body;
(b) monitoring the scattering properties of said opaque body so as to produce a monitor signal;
(c) utilising fluctuations in said monitor signal over time to infer fluctuations in said opaque body.

26. A method as claimed in claim 25 wherein said body comprises a human body.

27. A method as claimed in claim 26 wherein said fluctuations include alterations in the blood flow rate within the human body.

28. A method as claimed in claim 25 wherein said fluctuations include alterations in the respiration rate in the human body.

29. A method as claimed in claim 25 wherein said low power microwave emitter is located adjacent the chest of the human body.

30. A method as claimed in claim 25 wherein said low power microwave emitter includes two antennas, one for output and one for input.

31. A method as claimed in claim 25 wherein said low power microwave emitter includes only one antenna.

32. A remote monitoring system for monitoring a series of patients at remote locations, said monitoring systems comprising:
(a) a series of portable monitoring units for monitoring fluctuations in a human, the monitoring units including at least one low power microwave emitter for locating adjacent the human body, a microwave detector for detecting in the scattering characteristics from the human body; a signal processing means for analysing said fluctuations in the power so as to thereby derive characteristics about said body, and a wireless communications interface for communication characteristics about said body with a spatially separated base station;
(b) a series of base stations, each further interconnected with an information distribution network, said base stations receiving said characteristics from said portable monitoring units and forwarding them to a centralised computing and storage resource;
(c) a centralised computing and storage resource for storing and monitoring said characteristics.

33. A system as claimed in claim 32 wherein said system further includes analysis means for analysing said characteristics for predetermined behaviours and raising a notification alarm upon the occurrence of said predetermined behaviours.
